(19) United States
(12) Patent Application Publication
(10) Pub. No.: US 2026/0137551 A1
(43) Pub. Date: May 21, 2026

(54) Device for infrared (IR) and near-infrared (NIR) light therapy with cold water immersion

(71) Applicant: Nick Reed, Sheridan, WY (US)

(72) Inventor: Nick Reed, Sheridan, WY (US)

(21) Appl. No.: 19/060,753

(22) Filed: Feb. 23, 2025

Related U.S. Application Data
(63) Continuation-in-part of application No. 18/955,946, filed on Nov. 21, 2024.

(51) Int. Cl. A61F 7/00; A61N 5/06

(52) U.S. Cl. CPC A61F 7/0053; A61F 7/007; A61F 7/0085; A61N 5/0616; A61F 2007/0003; A61F 2007/0052; A61F 2007/0056; A61F 2007/0075; A61F 2007/008; A61F 2007/0086; A61F 2007/0095; A61N 2005/0632; A61N 2005/0636; A61N 2005/066

(57) ABSTRACT

The present invention integrates infrared (IR) and near-infrared (NIR) light therapy with cold water immersion, providing combined therapeutic benefits for skin health, stress reduction, and enhanced healing. This variation utilizes the water's scattering and heat-conductive properties to optimize the distribution and safety of infrared light while maintaining the cooling benefits of the device.

### BACKGROUND OF THE INVENTION

The present invention relates generally to devices and systems for cold therapy. It finds utility in the health, wellness, and beauty industries, providing users with improved access to the physiological and aesthetic benefits of targeted cold therapy.

### BRIEF SUMMARY OF THE INVENTION

The present invention integrates infrared (IR) and near-infrared (NIR) light therapy with cold water immersion, providing combined therapeutic benefits for skin health, stress reduction, and enhanced healing. This variation utilizes the water's scattering and heat-conductive properties to optimize the distribution and safety of infrared light while maintaining the cooling benefits of the device.

### BRIEF DESCRIPTION OF THE FIGURES

FIG. 1: illustrates one embodiment of an IR and NIR light therapy cold water immersion device.

FIG. 2: illustrates another embodiment of an IR and NIR light therapy cold water immersion device.

### DETAILED DESCRIPTION

There are two embodiments of the present invention disclosed herein: a facial cold plunge with an IR and NIR light therapy feature, and a body cold plunge with an IR and NIR light therapy feature (which might or might not include a facial cold plunge feature).

A unique feature of the invention is the retractable breathing apparatus, which allows users to sustain facial submersion for extended durations without the need to interrupt the immersion process to breathe. This apparatus comprises a snorkel-like breathing tube constructed of medical-grade silicone, which is mounted on the housing unit adjacent to the reservoir.

The present invention further includes a variation designed to attach to the wall of a full-body cold plunge, enabling the integration of facial immersion therapy into an existing system. In this configuration, the device features an adaptable mounting mechanism that securely affixes the ergonomic reservoir to the rim or sidewall of a full-body plunge tub.

A submersible pump, operatively connected to the main unit, draws water from the full-body plunge tub into the facial immersion bowl and recirculates it back into the tub. This closed-loop circulation system eliminates the need for separate chilling and filtration mechanisms within the facial device itself, as the water is maintained at the desired temperature and cleanliness by the full-body plunge's primary systems.

The tube is mechanically retractable via a spring-based or tensioned arm mechanism, allowing it to be stowed when not in use. The breathing tube is further equipped with a two-way check valve system that regulates airflow, preventing water ingress while facilitating effortless inhalation and exhalation during submersion. The height and position of the breathing tube can be adjusted to align with the user's facial orientation, enhancing comfort and usability.

The breathing apparatus, ergonomic reservoir, and optional UV sterilization components remain integral to this variation, ensuring users experience the same therapeutic and cosmetic benefits while leveraging existing equipment. This configuration enhances the versatility and cost-effectiveness of the invention, making it accessible to users who already utilize full-body cold plunge systems but desire the convenience and specificity of facial cold therapy.

In this configuration, the ergonomic bowl-shaped reservoir is equipped with infrared and near-infrared light sources embedded in a light pad positioned at the base of the bowl. The light pad emits IR and NIR light through the water, with the water serving as a medium to scatter the light evenly across the submerged face. This design ensures uniform exposure and prevents the light from escaping beyond the waterline. The light pad is backed by a heat-resistant insulating layer to prevent overheating of the components and condensation on the cold surface of the bowl. The light intensity is carefully calibrated to leverage the water's thermal conductivity, allowing for the safe use of higher intensity levels than conventional air-based systems.

The infrared system is operably connected to a power source equipped with a timer to limit session durations, ensuring user safety while maintaining therapeutic efficacy. The user activates the lights via a power button located on the device. Alternatively, an optional snorkel trigger mechanism may be included, which automatically activates the lights when the snorkel is submerged and deactivates them when the snorkel returns to its default position.

The design also incorporates specific features to protect the eyes from direct infrared exposure. A scuba mask-like seal surrounds the eyes, ergonomically fitting the user's face to block light while leaving the eye area exposed to cold water for puffiness reduction. The combination of cold water and infrared exposure reduces the traditional 20-30 minute therapy duration to just a few minutes, enhancing convenience while maintaining effectiveness.

This enhanced variation is particularly beneficial for users with sensitive skin who may experience dryness or redness from traditional infrared therapy. The cold water circulation, maintained by the integrated chilling and filtration system, ensures that any localized heating effects from infrared exposure are mitigated, preserving the skin's health while amplifying therapeutic benefits.

Additionally, the invention includes a larger variation designed for integration into a full-body cold plunge system. Infrared and near-infrared light pads are installed along the side walls of the plunge tub, with light shining through perforations in the liner. This setup utilizes the cooling effects of the water to safely accommodate higher light intensity levels, enabling users to experience enhanced benefits of simultaneous cold and infrared therapy in reduced session times. The system includes a GFCI-protected power source and a timer to ensure safety and optimal use.

These innovations make the infrared-integrated facial cold plunge device and its full-body variation uniquely positioned to deliver dual therapeutic benefits efficiently, addressing issues of skin sensitivity, overheating, and prolonged therapy durations that are common with traditional infrared systems.

The bowl incorporates infrared (NIR) and red lights that shine through a light pad positioned at the base of the bowl.
Lights are terminated at the waterline, as water scatters the infrared and red light, preventing light from escaping and ensuring even distribution across the submerged face.
The bowl's shape is ergonomically designed to maintain an equidistant relationship between the user's face and the light sources for uniform exposure.

2. Light and Heat Management:
The light pad is backed by a heat-resistant and insulating pad to prevent overheating of the components and to minimize condensation from the cold bowl.
The light pad is connected to a power supply with a timer, ensuring a short, safe duration of operation.

3. Activation Mechanism:
A power button located on the device allows users to manually activate or deactivate the lights after submersion.
An optional auto on/off snorkel trigger mechanism may be included:
Activates lights when the snorkel is pushed into the water during submersion.
Deactivates lights when the snorkel is removed or springs back up after the user's face is lifted.

4. Eye Protection:
A soft, oval-shaped scuba mask-like seal inside the bowl ergonomically fits around the eyes, blocking infrared light from directly exposing the eyes.
The eye area remains exposed to cold water for puffiness reduction benefits.

5. Cold Water Circulation:
The water is chilled and circulated by an integrated chiller and pump, which prevents overheating effects caused by infrared exposure.
The thermal conductivity of water allows for the safe use of higher infrared intensity levels, reducing therapy time significantly compared to air-based systems.

6. User Benefits:
Enables users with sensitive skin (who may experience drying or redness from traditional infrared therapy) to access infrared therapy benefits safely.
Reduces the standard 20-30 minute infrared therapy duration to a matter of minutes.
Combines the benefits of cold exposure and infrared therapy, improving skin health and reducing puffiness while offering deeper therapeutic effects.

### 2. Near-infrared Cold Plunge

### Key Features and Functionality:

1. Integrated Infrared Technology:
The cold plunge tub incorporates near-infrared (NIR) and red light therapy pads installed along the side walls, with light shining through perforations in the liner.
The light intensity levels are higher than what is safely possible in air-based systems due to the cooling effects of the water.

2. Power and Safety:
The light pads are connected to a power source with a timer and GFCI protection for user safety.
The timer limits session duration for safety and ensures proper therapeutic use.

3. Therapeutic Efficiency:
The combination of cold therapy and NIR light therapy allows users to experience enhanced benefits simultaneously.
The water's heat conductivity and scattering properties allow for efficient infrared therapy in a shorter period, maximizing therapeutic outcomes.

4. Applications:
Addresses the needs of individuals who may not tolerate traditional infrared therapy due to overheating or skin sensitivity.
Provides efficient access to combined cold and infrared therapy benefits, reducing session times while enhancing effectiveness.

### Summary of Key Innovations:

1. Utilization of water's properties (scattering and thermal conductivity) to enhance the efficacy and safety of infrared therapy.
2. Combination of cold and infrared therapies in a single device for dual benefits.
3. Reduced therapy duration with higher intensity levels of infrared light within safe limits.
4. Solutions for skin sensitivity, overheating, and overexposure issues commonly associated with traditional infrared devices.

### DETAILED DESCRIPTION OF FIGURES

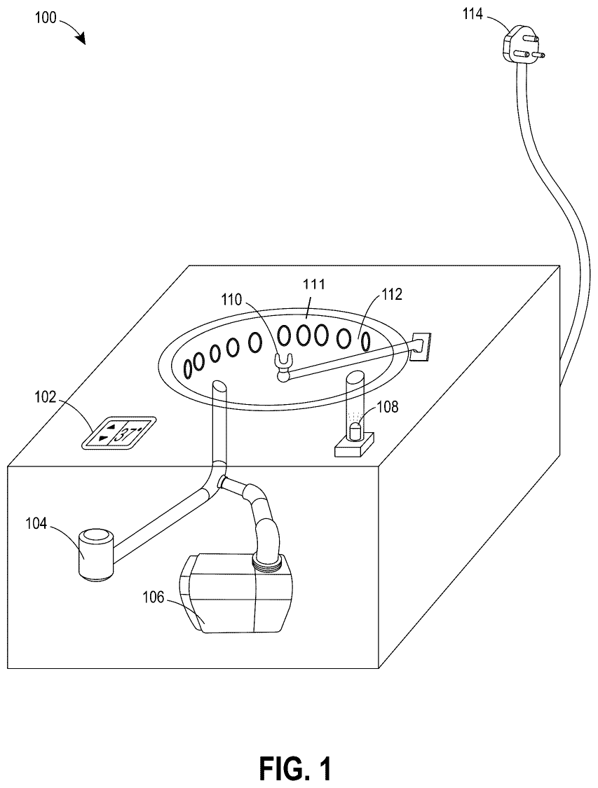

FIG. 1: illustrates the structural and functional components of the facial cold plunge device, showcasing its innovative features that facilitate effective and user-friendly operation. The following components are labeled with their respective reference numerals:

### 102: Adjustable Thermostat

The adjustable thermostat, integrated into the device, allows users to precisely set and maintain the desired water temperature. The thermostat includes a digital interface displaying real-time temperature readings and programmable controls for custom settings.

### 106: Filter

The filtration system, identified as component 106, ensures water hygiene by removing impurities, bacteria, and debris. The filter is replaceable and part of the closed-loop water circulation system, maintaining the cleanliness of the water for multiple uses.

### 108: UV Light

Positioned adjacent to the filtration system, the UV light sterilizes the circulating water by eliminating microbial contaminants, further enhancing water purity and safety during prolonged immersion.

### 110: Removable Mouthpiece for Easy Cleaning

The mouthpiece of the breathing apparatus, designated as 110, is detachable to facilitate thorough cleaning. Constructed from medical-grade silicone, it ensures hygiene and comfort during repeated usage.

### 111: Light Therapy Bulbs

The facial cold plunge incorporates near-infrared (NIR) and red light therapy pads installed along the side walls, with light shining through perforations in the liner. The light intensity levels are higher than what is safely possible in air-based systems due to the cooling effects of the water.

### 112: Lowering Attached Breathing Mechanism

The attached breathing mechanism, referenced as 112, is a retractable snorkel-like tube that lowers into position for use. This component includes an adjustable height mechanism and a two-way check valve to allow seamless respiration while preventing water ingress during facial immersion. A soft, oval-shaped scuba mask-like seal inside the bowl ergonomically fits around the eyes, blocking infrared light from directly exposing the eyes. The eye area remains exposed to cold water for puffiness reduction benefits.

### 114: GFCI Outlet

The Ground Fault Circuit Interrupter (GFCI) outlet, labeled 114, ensures electrical safety during operation, protecting the user and device from potential electrical faults, particularly in wet environments.

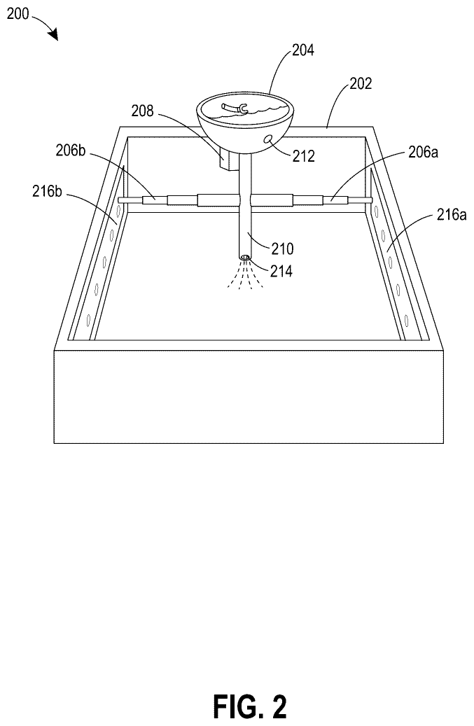

FIG. 2 illustrates a second embodiment.

### 200: Integrated Assembly of Cold Plunge Tub and Facial Plunge Device

In this schematic, the facial cold plunge device sits in a cold plunge tub which contains its own water reservoir.

### 202: Cold Plunge Tub

The cold plunge tub can be purpose built to operate with the facial cold plunge or the facial cold plunge can be retrofitted into an existing cold plunge tub.

### 204: Facial Cold Plunge Lowering Attached Breathing Mechanism

The attached breathing mechanism is a retractable snorkel-like tube that lowers into position for use. This component includes an adjustable height mechanism and can contain a two-way check valve to allow seamless respiration while preventing water ingress during facial immersion.

### 206a and 206b: Telescoping Legs

The telescoping legs are configured to make the facial cold plunge device adjustable for a variety of cold plunge tub designs as well as adjustable. They can extend and retract to position the facial cold plunge suitably for operation by a user sitting in the cold plunge tub.

### 208: Pump

A submersible pump, operatively connected to the main unit, draws water from the full-body plunge tub into the facial immersion bowl and recirculates it back into the tub. This closed-loop circulation system eliminates the need for separate chilling and filtration mechanisms within the facial device itself, as the water is maintained at the desired temperature and cleanliness by the full-body plunge's primary systems.

### 210: Inlet Pipe

The inlet pipe carries water from the cold plunge tub reservoir. The circulation system may also incorporate insulated conduits to prevent thermal losses during operation, ensuring consistent and efficient cooling.

### 212: Outlet Opening

Water from the facial cold plunge is exhausted through the outlet opening. In some embodiments, there may be a conduit from the outlet opening to either an external drain for the water to exit the system or to a filtration unit with the cold plunge tub for filtration before return to the cold plunge tub reservoir.

### 214: Inlet Opening

Water from the cold plunge tub reservoir enters through the inlet opening and flows up the inlet pipe due to the suction force provided by the pump.

### 216a and 216b: Light Therapy Bulbs.

The cold plunge tub incorporates near-infrared (NIR) and red light therapy pads installed along the side walls, with light shining through perforations in the liner. The light intensity levels are higher than what is safely possible in air-based systems due to the cooling effects of the water.

What is claimed is:

1) A cold plunge device with a infrared (IR) and near-infrared (NIR) light therapy feature, comprising:
a) a cold plunge housing with infrared and near-infrared light sources embedded in a light pad positioned along a sidewall;
b) an ergonomically contoured, bowl-shaped reservoir integrated into the housing unit and configured to accommodate a user's face for water immersion with infrared and near-infrared light sources embedded in one or more light pads positioned at the base of the bowl;
c) a chilling mechanism operatively connected to the reservoir, the chilling mechanism comprising evaporative cooling coils and a compressor or thermoelectric cooling system for maintaining water temperature at a user-defined level below 50° F.;
d) a thermostat control system operably connected to the chilling mechanism, the thermostat system comprising a user interface with a digital display and programmable temperature settings for precise control of water temperature;
e) a water circulation system, comprising:
i) a pump configured to circulate water within the reservoir;
ii) insulated conduits connecting the pump to the reservoir; and
iii) a filtration assembly including a replaceable filter configured to remove impurities and maintain hygienic water conditions.
f) a retractable breathing apparatus mounted to the housing unit, the breathing apparatus comprising:
i) a snorkel-like breathing tube constructed from medical-grade silicone;
ii) a two-way check valve for regulating airflow and preventing water ingress; and
iii) an adjustable positioning mechanism for aligning the breathing tube with a user's facial orientation during water immersion; and
g) a power source operatively connected to the chilling mechanism, pump, and thermostat control system, the power source configured to enable continuous operation.

2) The facial cold plunge device of claim 1, wherein the reservoir is contoured to provide a watertight seal against the user's face to minimize water spillage during use.

3) The facial cold plunge device of claim 1, wherein the chilling mechanism is operable to cool water within the reservoir to temperatures as low as 32° F. while maintaining uniform cooling across the reservoir surface.

4) The facial cold plunge device of claim 1, wherein the filtration assembly comprises a carbon or HEPA filter capable of removing bacteria, debris, and other impurities from the water during operation.

5) The facial cold plunge device of claim 1, wherein the retractable breathing apparatus is operatively mounted on a spring-loaded mechanism to allow stowing when not in use and deployment during immersion.

6) The facial cold plunge device of claim 1, wherein the two-way check valve of the breathing apparatus is configured to allow seamless inhalation and exhalation while submerged in water.

7) The facial cold plunge device of claim 1, wherein the thermostat control system includes a programmable timer function to automatically maintain the desired water temperature over a predefined period.

8) The facial cold plunge device of claim 1, wherein the housing unit comprises a compact, lightweight design with rubberized feet or suction cups for enhanced stability during operation.

9) The facial cold plunge device of claim 1, further comprising an optional battery-powered mode to enable portable and off-grid use.

10) The facial cold plunge device of claim 1, wherein the water circulation system is configured to operate continuously to maintain consistent water temperature and filtration during extended periods of use.

11) The facial cold plunge device of claim 1, wherein the breathing apparatus is adjustable in length and height to accommodate users of varying facial dimensions and preferences.

12) The facial cold plunge device of claim 1, wherein the ergonomic reservoir is constructed with non-slip interior coatings to enhance user safety and comfort during immersion.

13) The facial cold plunge device of claim 1, wherein the digital display of the thermostat system provides real-time feedback on water temperature and system status.

14) The facial cold plunge device of claim 1, further comprising an integrated safety mechanism configured to shut down the chilling mechanism and pump in the event of water leakage or overheating.

15) The facial cold plunge device of claim 1, wherein the device is configured to support detachable accessories, including alternative filtration systems and enhanced breathing apparatus configurations.

16) A variation of the facial cold plunge device of claim 1, wherein the device is configured to attach to the wall of a full-body cold plunge system, comprising:
a) a mounting mechanism adapted to secure the reservoir to the rim or sidewall of the full-body cold plunge tub;
b) a submersible pump operatively connected to the reservoir, configured to draw water from the full-body plunge tub into the reservoir and return it, thereby utilizing the primary chilling and filtration systems of the full-body plunge; and
c) a closed-loop circulation system enabling continuous water flow between the full-body cold plunge tub and the reservoir, while maintaining ergonomic and respiratory functionalities of the facial cold plunge device.

17) The facial cold plunge device of claim 16, wherein the mounting mechanism comprises two or more telescoping legs.
